(19) United States
(12) Patent Application Publication
(10) Pub. No.: US 2026/0216469 A1
(43) Pub. Date: Jul. 30, 2026

(54) MEDICAL VENTILATOR WITH OPERATED PEP VALVE

(71) Applicant: Air Liquide Medical Systems, Antony (FR)

(72) Inventor: Taraq BECHIKHI, Livry-sur-Seine (FR)

(21) Appl. No.: 19/455,062

(22) Filed: Jan. 21, 2026

(30) Foreign Application Priority Data
Jan. 30, 2025 (FR) FR2500983

(51) Int. Cl. A61M 16/20; A61M 16/00

(52) U.S. Cl. CPC A61M 16/205; A61M 16/0066

(57) ABSTRACT

The invention relates to a medical ventilator (100) comprising a motorized turbine (41) for supplying a gas, the turbine being operated by a computer (40). A gas circuit (102) with an inspiratory branch (102.1) and an expiratory branch (102.2) in fluidic communication with the atmosphere. A PEP valve (1), arranged on the expiratory branch (102.2), comprises a valve shutter (2) collaborating with a valve seat (3). An electromechanical-control device (20), operated by computer (40), acts on the rear face (2.2) of the valve shutter (2) in order to push it towards the valve seat (3). A pressurizing chamber (22) in fluidic communication with a pressure-conveying line (42) is supplied by the turbine (41) and collaborates with the valve shutter (2).

### CROSS REFERENCE TO RELATED APPLICATIONS

This application claims the benefit of priority under 35 U.S.C. § 119 (a) and (b) to French Patent Application No. FR2500983, filed Jan. 30, 2025, the entire contents of which are incorporated herein by reference.

### BACKGROUND

The invention relates to an assisted-ventilation apparatus or medical ventilator with a PEP valve that is operated pneumatically and electromechanically.

Certain artificial respirators, also known as medical ventilators, used for ventilating individuals who need this, namely patients, with a respiratory gas such as air or an air/oxygen mixture, according to successive respiratory cycles, comprise an expiratory valve arranged in the pneumatic circuit thereof, for example on an expiratory branch of the pneumatic circuit, namely the gas circuit internal to the apparatus. Each respiratory cycle comprises:
an inspiratory phase during which the expiratory valve is completely closed so as to allow the gas coming from the medical ventilator to enter the patient's lungs.
an expiratory phase during which the expiratory valve opens to allow the patient to exhale to outside the pneumatic circuit, either at atmospheric pressure or at a pressure higher than atmospheric pressure.

The expiratory valve serves to allow the patient to exhale gas at a pressure above atmospheric pressure, known as a Positive Expiratory Pressure (PEP) or PEP pressure. In order to achieve this, the expiratory valve or PEP valve needs to be controlled such that it can be either completely closed (in the inspiratory phase) or at least partially open (in the expiratory phase) in order to empty the pneumatic circuit and also maintain a PEP pressure in the pneumatic circuit.

At the present time, PEP valves can be controlled only pneumatically. Thus, in the inspiratory phase, a control gas-pressure equal to the pressure in the pneumatic circuit is applied to the PEP valve in order to allow it to close fully, whereas in the expiratory phase, a gas pressure lower than the inspiratory pressure, calculated and set by the respirator operating means, for example an electronic board with a microprocessor, is applied to the PEP valve so as to allow it to open to empty and depressurize the pneumatic circuit and maintain the desired PEP pressure therein.

Respirators, i.e. respiratory apparatuses, are equipped with a main pressure or flow source for pressurizing the pneumatic circuit and applying the desired pressure to the patient. The main pressure or flow source may for example be a motorized turbine, also referred to as a compressor, a (micro)blower or the like, controlled by the respirator operating means. The terms turbine, compressor and (micro)blower are considered to be equivalent and interchangeable.

The motorized turbine may act as a source of pressure used for controlling the expiratory valve, i.e. the PEP valve. In that case:
in the inspiratory phase, the electric motor of the turbine is controlled to make it accelerate, supply the respiratory gas, and thus pressurize the pneumatic circuit and deliver the desired inspiratory pressure or flow to the patient. The control pressure for the PEP valve is then equal to that of the gas leaving the turbine and therefore to that prevailing in the patient's pneumatic circuit, enabling the PEP valve to close fully.

in the expiratory phase, a pressure lower than the inspiratory pressure, calculated and set by the respirator, is applied to the PEP valve so as to allow it to open to empty the pneumatic circuit and maintain the pressure known as PEP pressure therein. The turbine decelerates, i.e. slows, and supplies a very low gas flow rate, referred to as “flow by”, to allow regulation of the PEP pressure in the patient circuit. The PEP pressure control comes either from the pressure supplied by the turbine or from another pressure source. The PEP pressure control switchover from the inspiratory phase to the expiratory phase is provided by electrovalves.

However, controlling the PEP valve purely pneumatically presents disadvantages and problems, notably a fairly long response time which prevents the patients from being ventilated at a high breathing rate; the creation, on exhalation, of a pneumatic brake the significance of which varies according to the settings of the machine; and, when the set Pep pressure is high, i.e. higher than approximately 10 hPa, the PEP becomes more difficult to regulate and the PEP valve starts to create, in the circuit, pneumatic vibrations that are fairly audible, and cause the respirator triggering system to malfunction.

In an attempt to overcome this, the use of an electromechanical control employing a proportional electromechanical actuator, known as a VCA (Voice Coil Actuator or mobile coil actuator) that acts mechanically on the valve shutter of the PEP valve in order to unseat it, or not unseat it, from its seat and thus open or close the PEP valve, as illustrated in FIG. 1, has been proposed.

While this solution avoids the abovementioned problems, it presents a major disadvantage, namely that of causing an electrical-current consumption that is far greater than that of a pneumatically controlled system, and this is unacceptable.

One problem is therefore that of being able to provide a ventilator having a PEP valve that is operated by an improved control system able to maintain the advantages of an electromechanical control system while at the same time appreciably reducing the resulting electrical power consumption and also without encountering the problems and disadvantages of a control system that is purely pneumatic.

### SUMMARY

One solution according to the invention relates to a medical ventilator, i.e. a respirator, comprising a motorized turbine for supplying a gas; operating means for operating the turbine; a gas circuit comprising an inspiratory branch for carrying the gas coming from the turbine, and an expiratory branch in fluidic communication with the atmosphere; and a PEP valve, arranged on the expiratory branch, comprising a valve shutter comprising a front face collaborating with a valve seat, and a rear face on the opposite side to the front face.

In addition, the medical ventilator further comprises an electromechanical-control device, operated by the operating means, acting on the rear face of the valve shutter in order to push it towards the valve seat, and a pressurizing chamber in fluidic communication with a pressure-conveying line supplied by the turbine, said pressurizing chamber collaborating with the rear face of the valve shutter.

Depending on the embodiment considered, the ventilator of the invention may comprise one or more of the following features:
the electromechanical-control device acts on the rear face of the valve shutter via an actuator.
the pressurizing chamber is arranged on the same side as the rear face of the valve shutter and is delimited by at least part of the surface of said rear face of the valve shutter.
the valve shutter is made from a supple material, preferably silicone or any other suitable material.
the valve shutter comprises an internal chamber forming the pressurizing chamber.
the electromechanical-control device comprises a coil and a magnet these being mobile one relative to the other.
the coil is controlled by the operating means.
the pressure-conveying line comprises an atmospheric-venting duct arranged between the turbine and the PEP valve.
the atmospheric-venting duct is equipped with a calibrated orifice.
the operating means comprise at least one (micro)processor.
the operating means comprise at least one electronic board with a microprocessor.
it comprises electrical power-supply means supplying electrical power to the operating means, to the motorized turbine and/or to the electromechanical-control device.
the gas circuit comprises passages, ducts or the like.

The invention also relates to a method for ventilating an individual who needs this, i.e. a patient, with a respiratory gas, such as air or an air/oxygen mixture (i.e. O2-enriched air), supplied by a medical ventilator according to the invention, according to successive respiratory cycles comprising an inspiratory phase during which the expiratory valve (i.e. the PEP valve) is controlled or operated so that it is completely closed so as to allow the gas coming from the medical ventilator to enter the patient's lungs (and therefore prevent it from being discharged to outside the pneumatic circuit); and an expiratory phase during which the expiratory valve (i.e. the PEP valve) is controlled or operated so that it opens to allow the patient to exhale to outside the pneumatic circuit.

### Definitions In the context of the invention:

the terms “duct”, “tube”, “pipe” or “line” are considered to be equivalent and interchangeable.
the terms “assisted-ventilation apparatus”, “medical ventilator” and “artificial respirator” are considered to be equivalent and interchangeable.
the terms “turbine”, “compressor” and “blower” are considered to be equivalent and interchangeable.
the terms “means of/to/for” are considered to be wholly equivalent to and capable of being interchanged with the terms “device of/to/for”; for example the term “operating means” may be replaced by “operating device”, the term “electrical power supply means” may be replaced by “electrical power supply device”, etc.
a “pressure measurement” means a pressure value (e.g. a numerical value) or a signal representative of such a pressure value, which reflects or corresponds to the gaseous pressure measured by a pressure sensor or the like.
a “flowrate measurement” means a flowrate value (e.g. a numerical value) or a signal representative of such a flowrate value, which reflects or corresponds to a flowrate measured by a flowrate sensor, such as a mass flowrate sensor, or to pressure values measured by a differential-pressure sensor or the like and then processed and/or converted into a flow rate for example inside operating means.
the terms “upstream” and/or “downstream” are used in relation to the normal direction of gas flow, namely in the direction leading towards the patient.

### BRIEF DESCRIPTION OF THE DRAWINGS

The invention will now be better understood from the following detailed description, provided by way of non-limiting illustration, with reference to the appended figures, in which:

FIG. 1 schematically depicts an electromechanical-control system using a proportional electromechanical actuator according to the prior art able to act on a medical ventilator PEP valve.

FIG. 2 schematically depicts one embodiment of a PEP valve for a medical ventilator according to the invention.

FIG. 3 schematically depicts a first embodiment according to the invention of a system for controlling the PEP valve of FIG. 2.

FIG. 4 schematically depicts a second embodiment according to the invention of a system for controlling the PEP valve of FIG. 2.

FIG. 5 schematically depicts a third embodiment according to the invention of a system for controlling the PEP valve of FIG. 2.

FIG. 6 schematically depicts the architecture and operation of a medical ventilator with a PEP valve.

### DETAILED DESCRIPTION OF PREFERRED EMBODIMENTS

FIG. 6 schematically depicts the architecture and operation of a ventilation apparatus or medical ventilator 100 comprising a PEP valve 1. The medical ventilator 100 comprises an outer casing or shell 101, for example made of polymer, in which are arranged the various components of the apparatus 100, notably the motorized turbine 41, i.e. (micro-)blower, compressor or the like, that draws ambient air in through an air inlet 41.1 and that is also fluidically connected, at outlet, to the internal gas circuit 102 of the ventilator 1 which here comprises an inspiratory branch 102.1 and an expiratory branch 102.2 equipped with a PEP valve 1, typically gas ducts or passages.

The motorized turbine 41 supplies the pressurized gas to the inspiratory branch 102.1 of the internal gas circuit 102 which carries it towards the patient P. The inspiratory branch 102.1 and expiratory branch 102.2 are connected to an external patient circuit 103, such as supple tubes joined at a Y-piece 105 able to carry the gas, e.g. air, coming from the inspiratory branch 102.1 as far as a respiratory interface 104, such as a breathing mask, used to supply the gas to the patient P and, moreover, to remove the exhaled gas (Gₑₓₚᵢ) exhaled by the patient P to the expiratory branch 102.2 which enables the CO2-rich exhaled gas to be discharged to the atmosphere.

Typically, the respiratory gas supplied by the turbine 41 is air, or oxygen-enriched air or even pure oxygen, notably according to the therapy/ventilation to be implemented. The oxygen may be supplied by an external source, for example a wall outlet supplied by a gas pipe or by an oxygen cylinder that supplies the ventilator 100 with additional oxygen,

for example upstream of the turbine 41 (and not depicted). The turbine 41 is motorized, that is to say it comprises an electric motor, typically a brushless motor with a rotational speed that can reach as high as 40,000 rpm, to 70,000 rpm, and preferably also having a low inertia. Classically, such a turbine 41 comprises an electric motor which is protected by a housing and, during its operation, drives an axle or rotary shaft carrying a bladed wheel arranged in the internal compartment of a volute casing which surmounts the electric motor and the housing. The volute casing comprises a gas inlet through which the air drawn in enters the internal compartment, and a gas outlet via which the pressurized gas leaves the internal compartment and is then supplied to the inspiratory branch 101.2 of the internal gas circuit 102. The volute casing can be surmounted by a cowl.

In order to control the circulation of gas in the internal gas circuit 102, particularly in the inspiratory branch 101.2 towards the patient P, various additional elements, such as one or more electrovalve(s), one or more pressure sensor(s) and/or flowrate sensor(s) or the like may be provided.

Also provided are operating means 40 which receive and process the pressure and/or flowrate measurements taken by the sensors, and also operate the electrovalve(s) and/or govern the accelerations and decelerations of the motor of the turbine 41 according to the respiratory phases of the patient P.

The operating means 40 advantageously comprise one or more microprocessor(s) executing one or more algorithm(s), preferably one or more microcontroller(s). Typically, the one or more microprocessor(s) are arranged on one or more electronic board(s).

More specifically, the turbine 41 is operated by the operating means 40, for example via a direct-voltage value generated with the aid of an all-or-nothing signal, typically being operated using Pulse-Width Modulation (PWM) control, or using a current value, and is regulated by monitoring the pressure, notably, via the pressure data fed back by a pressure sensor.

The operating means 40 may also comprise and/or collaborate with memory-storage means, such as one or more datalogging memories, for example a high-speed memory, i.e. a flash memory, acting as a read-only memory, and also a random access memory known as RAM.

The flash memory makes it possible to record and preserve the parametrization of the ventilation modes, that is to say the pressure, flowrate, etc. values associated with each ventilation mode, and other data, information items, operating parameters or other parameters that need to be retained in the apparatus 100. The stored ventilation modes are, for example, a CPV mode, a CPAP mode, PACV, ASB, HFOT or any other ventilation mode.

Moreover, the RAM memory makes it possible to temporarily store any parameter modifications that are desired by the user, or any other data, information items or temporary parameter. These modifications can be entered in the ventilator 100 by means, for example, of a graphical user interface (GUI) or a human machine interface (HMI) comprising setting means (e.g. buttons, keys, cursors or others) and a graphical display screen in colour or in black and white. The graphical display screen may be a touch screen, and the setting means of the GUI or of the HMI serving to modify or adjust the ventilation settings (i.e. parameters) are then preferably virtual or analog keys displayed on said touch screen.

Electrical power supply means 106 supply power to the components of the ventilator 100 that require an electrical current in order to function, for example the turbine 41, the operating means 40, the HMI, the sensors, etc. The electrical power supply means 106 may comprise a 110/220 V mains power supply (e.g. cable, electrical outlet, etc.) and/or an internal rechargeable battery.

Finally, it must be emphasized that the circulation of gas in the expiratory branch 102.2 is controlled by an expiratory valve, namely a PEP valve 1, arranged on said expiratory branch 102.2 of the ventilator 100, as detailed hereinafter.

The ventilator 1 is configured to operate according to respiratory cycles, where each respiratory cycle comprises:
an inspiratory phase during which the expiratory valve 1 is completely closed so as to allow the gas coming from the turbine 41 of the medical ventilator 100 to be conveyed as far as the patient P by the inspiratory branch 102.1, the patient circuit 103 and the respiratory interface 104 and then enter the lungs of the patient P.
an expiratory phase during which the expiratory valve 41 opens so as to allow the patient to exhale, either at atmospheric pressure or at a pressure higher than atmospheric pressure (i.e. PEP pressure). The gas exhaled by the patient into the interface 104 therefore passes via the patient circuit 103, the expiratory branch 102.2 of the ventilator 100 and the PEP valve 1, before being discharged into the surrounding atmosphere.

The PEP valve 1 therefore needs to be controlled or operated to ensure that it opens or closes according to the successive respiratory cycles.

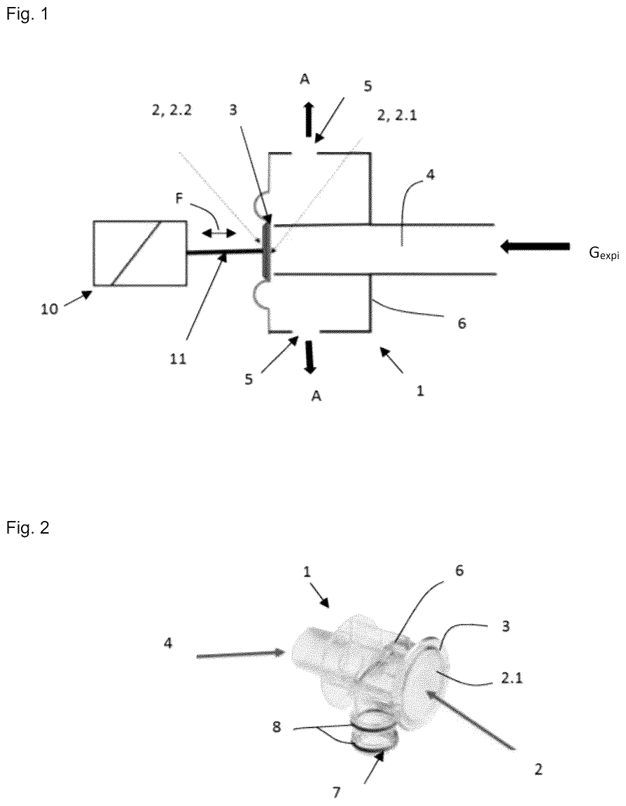

Thus, FIG. 1 schematically indicates an electromechanical-control system for controlling the expiratory valve or PEP valve 1 of a medical ventilator 100 such as that of FIG. 6 by means of a proportional electromechanical actuator 10, 11 comprising an actuating stem 11, or VCA according to the prior art.

The actuator 10, 11 acts mechanically, via the stem 11, by applying a variable force (at F) to the rear face 2.2 of the valve shutter 2 of the PEP valve 1, to which it is secured, so that:
during the expiratory phases, the front face 2.1 of the valve shutter 2 is unseated from the valve seat 3 and the PEP valve 1 is thus opened so as to allow gas to escape to the atmosphere A via the outlet orifice(s) 5 formed in the valve body 6, this typically being the CO₂-rich exhaled gas (Gₑₓₚᵢ) carried by the supply duct 4 connected to the expiratory branch of the patient circuit, or
conversely, during the inspiratory phases, it is pushed back against the valve seat 3 and the PEP valve 1 is thus closed so as to block the escape of gas to the atmosphere A via the outlet orifice(s) 5 and respiratory gas can be supplied to the patient.

Now, the use of such an electromechanical control system 10 in a ventilator 100 such as that of FIG. 6 has the disadvantage of leading to a high consumption of electrical current.

FIG. 2 schematically indicates one embodiment of a PEP valve 1 according to the invention which is intended to be arranged on the expiratory branch 102.2 of the internal gas circuit, i.e. the pneumatic circuit, of a medical ventilator 100 such as that of FIG. 6 described hereinabove.

The PEP valve 1 according to the invention comprises a valve body 6 comprising a valve shutter 2 collaborating with a valve seat 3 to allow or prevent the passage of gas through the valve body 6 towards an outlet orifice 7 borne by an outlet duct 8 in fluidic communication with the surrounding atmosphere.

The valve body 6 further comprises a supply duct 4 for conveying the gas (Gₑₓₚᵢ) exhaled by the patient, which is rich in CO₂. This supply duct 4 is intended to be fluidically connected to the expiratory branch 102.2 of the ventilator 100 which is supplied with the gas exhaled by the patient P.

In this case, the gas pressure coming from the turbine 41 is applied to the valve shutter 2 to control the opening/closing of the PEP valve 1.

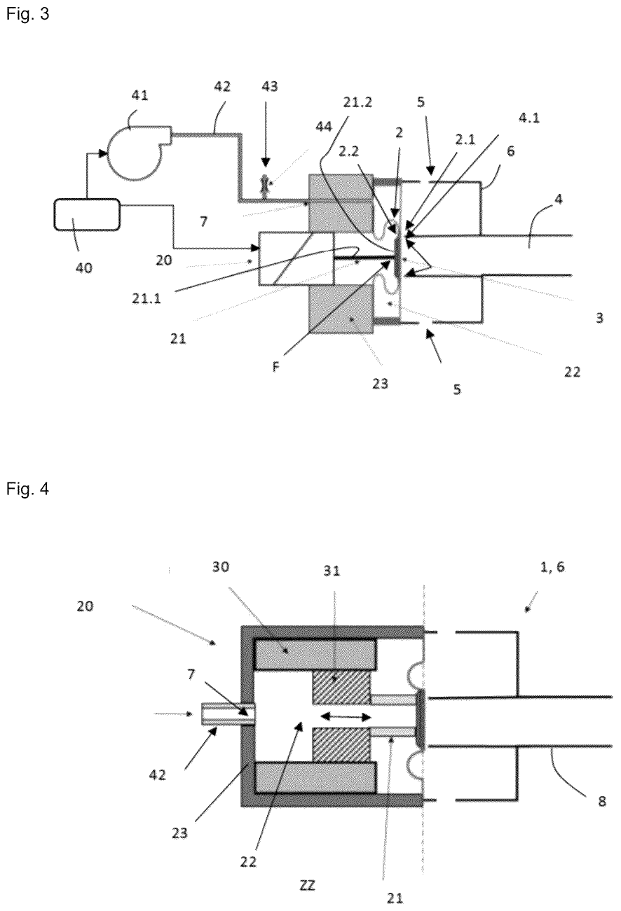

FIG. 3 schematically depicts a first embodiment of a hybrid control system 20, 42 for controlling a PEP valve 1 according to the invention, such as the valve 1 of FIG. 2, of a medical ventilator 100 such as that of FIG. 6 described hereinabove.

The hybrid control system 20, 42 according to the invention is based on dual control of the PEP valve 1 combining a pneumatic pressure conveyed by a pneumatic line 42 supplied with the gas supplied by the turbine 41, and an electromechanical force F acting on the rear face 2.2 of the valve shutter 2 of the valve 1 via an electromechanical control system comprising an actuator 20, 21 with a stem 21.1 for closing or opening the valve 1 proportionally, by pushing the valve shutter 2 back towards the valve seat 3 to a greater or lesser extent.

In other words, the hybrid control system 20, 42 associated with the PEP valve 1 of a ventilator 100 according to the invention comprises:
an electromechanical-control device 20, operated by operating means 40 of the ventilator 100, typically a (micro)processor of an electronic board, acting on the rear face 2.2 of the valve shutter 2 in order to push it towards the valve seat 3 by applying to it a force (F) that is proportional to the desired degree of opening of the PEP valve 1, during the expiratory phases, and
a pressurizing chamber 22 in fluidic communication with a pressure-conveying line 42 supplied by the turbine 41, for receiving pressurized gas, said pressurizing chamber 22 collaborating with the rear face 2.2 of the valve shutter 2 so as to apply a pneumatic, i.e. a gas pressure to it, as explained hereinafter.

The electromechanical-control device (i.e. system) comprises an actuator 20, 21 that acts against the rear face 2.2 of the valve shutter 2 in order to push it towards the valve seat 3. The actuator 20, 21 may be secured, i.e. fixed, to said rear face 2.2 of the valve shutter 2.

The actuator 20, 21 is moved by a proportional electromagnet (VCA).

The electromechanical-control device (i.e. system), particularly the actuator 20, 21, is at least partially also arranged inside a casing 23 that can be secured to the PEP valve body 6. The actuator 21 has the ability to move inside the casing 23.

In the embodiment of FIG. 3, the actuator 21 comprises a stem 21.1, such as a rod or the like, bearing a head 21.2, here in the form of a disc, secured to the rear face 2.2 of the valve shutter 2, for example by adhesive bonding or some other means.

The valve shutter 2 is preferably made of a material that is supple, i.e. deformable or flexible, for example of silicone or any other suitable material.

The front face 2.1 of the valve shutter 2 collaborates with the valve seat 3 comprising the terminal edge 4.1 of the duct 4 to control the passage of gas.

When the front face 2.1 of the valve shutter 2 comes to bear against the valve seat 3 the duct 4 is closed in a fluidtight manner that prevents any circulation of gas. Conversely, when the front face 2.1 of the valve shutter 2 is not pressing or is no longer pressing against the valve seat 3, the duct 4 is opened thus allowing gas to circulate towards the external atmosphere.

In other words, the stem 21.1 and the valve shutter 2 can be pushed or pulled depending on whether the PEP valve 1 is to be opened or closed.

In this first embodiment, the valve shutter 2 employed has a special structure because it comprises the pressurizing chamber 22, namely an internal pressurizing chamber, that receives the gas pressure conveyed by the pressure-conveying line 42, such as a duct or the like, so as to provide pneumatic operation of the valve 1.

The pressure-conveying line 42 comprises an atmospheric-venting duct 43, arranged between the turbine 41 and the valve 1, i.e. preferably a duct or passage equipped with a calibrated orifice 44. The atmospheric-venting duct 43 makes it possible to accelerate the depressurizing of the pressurizing chamber 22 of the PEP valve 1 upon the transition from an inspiratory phase to an expiratory phase.

The pressure-conveying line 42 may be fluidically connected to the internal chamber 22 of the valve shutter 2 via a canal 7 or the like passing through the wall of the casing 23.

During operation, particularly during the expiratory phases, the electromechanical-control device applies to the rear face 2.2 of the valve shutter 2, via the actuator 20, 21, a force F of greater or lesser magnitude, i.e. a proportional force, that is directed towards the seat 3 so as to close the PEP valve 1 to a greater or lesser extent, as explained hereinabove, which means to say that the force F applied is proportional to the PEP pressure.

As a result, during the expiratory phase, the turbine 41 slows and causes the pressure in the pressure-conveying line 42 to decrease, causing the pressure in the chamber to decrease with the leakage of gas via the atmospheric-venting duct 43 bearing the gas outlet orifice.

In the absence of pressure exerted in the pressurizing chamber 22 of the valve shutter 2, the expiratory valve, i.e. the PEP valve 1, can be closed by the force F that is directly proportional to the pressure prevailing in the pneumatic circuit of the patient, which pressure is given by the relationship:

Pressure=force F/ surface-area of valve seat.

Applying such a pressure to the valve shutter 2 requires consumption of electrical power on the part of the electromechanical-control device 20 and the greater the surface-area of the valve seat and/or the greater the pressure in the circuit, the greater this consumption will be.

Now, the gas pressure delivered by the turbine 41 and which is carried by the pressure-conveying line 42 and the canal 7 is applied directly to the pressurizing chamber 22 of the valve shutter 2 of the PEP valve 1, namely in this instance to the internal pressurizing chamber thereof.

This pneumatic pressure exerted in the pressurizing chamber 22 of the valve shutter 2 will also be applied to the rear face 2.2 of the valve shutter 2, in addition to the force F exerted by the actuator 21 of the electromechanical-control device 20, and this will enable a significant reduction in the force F that has to be applied by the electromechanical-control device 20 and therefore make it possible also to reduce the amount of energy consumed by this electromechanical-control device 20.

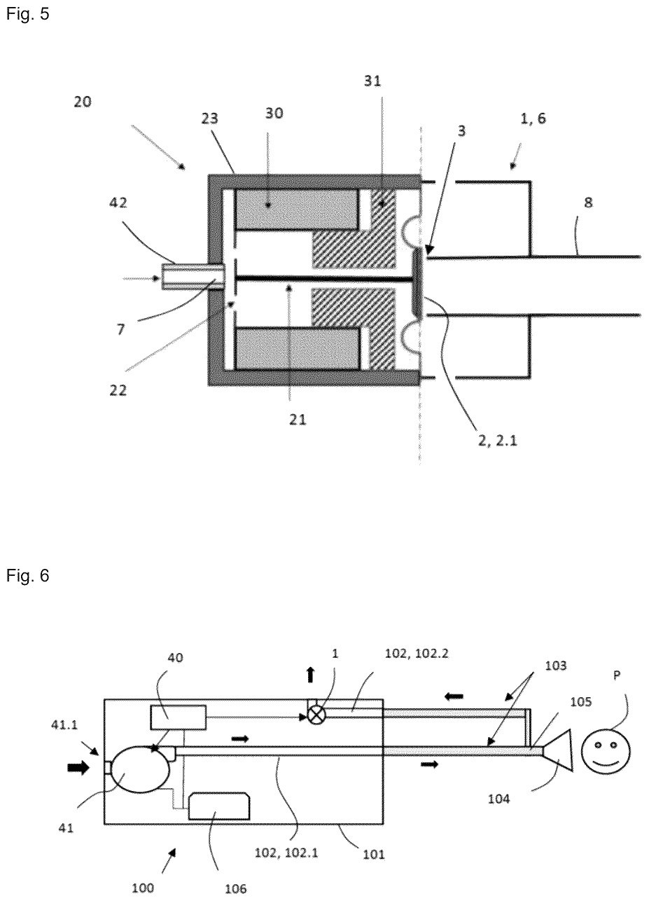

FIG. 4 and FIG. 5 schematically depict a second and third embodiment of a control system for controlling the PEP valve 1 of FIG. 2 according to the invention, which operate in accordance with the principle set out hereinabove and described in connection with the first embodiment of FIG. 3; those elements that are common to each are therefore not detailed again hereinafter.

In the second embodiment of FIG. 4, the electromechanical control device 20 comprises a fixed electrical coil 30 collaborating with a mobile magnet 31 bearing an actuator 21, such as an intermediate component bonded to the magnet, which actuator 21 is, once again, secured to the rear face 2.2 of the valve shutter 2 so as to be able to exert on said valve shutter 2 a proportional mechanical force as explained hereinabove.

The electrical coil 30 and the magnet 31 are arranged inside the casing 23.

The electrical coil 30 is controlled by the operating means 40 so as to cause the mobile magnet 31 to move translationally, notably towards the valve shutter 2. This is the principle of operation of a proportional electromagnet having a mobile magnet.

As already stated, the force applied to the actuator may be in the 2 directions, namely may enable the valve shutter of the PEP valve 1 to be pushed or pulled.

The electrical coil 30 consumes electrical current while it is in operation, as in the first embodiment.

Once again, there is a pressurizing chamber 22 supplied with gas pressure by the pressure-conveying line 42 supplied by the turbine 41 and collaborating with the rear face 2.2 of the valve shutter 2 so that the pressure prevailing therein can be exerted on the rear face 2.2 of the valve shutter 2, as already explained, here again making it possible to reduce the electrical power consumption of the coil 30.

In this case, the pressurizing chamber 22 is arranged in the casing 23 and is supplied with gas via a canal 7 passing through the wall, for example the rear wall, of the casing 23, which canal 7 is, as previously, supplied by the pressure-conveying line 42 that connects to it.

FIG. 5 schematically depicts a third embodiment according to the invention which is, on the whole, analogous to that of the second embodiment of FIG. 4, but operates in reverse.

Specifically, in this third embodiment, the electromechanical-control device 20 also comprises an electrical coil 30 collaborating with a magnet 31, but in this instance, the magnet 31 is fixed, i.e. immobile, while the electrical coil 30 is able to move translationally and bears the actuator 21 which is secured to the rear face 2.2 of the valve shutter 2 so as to be able to exert a proportional mechanical force on said valve shutter 2, as explained hereinabove.

As in the second embodiment, a pressurizing chamber 22 supplied with gas pressure by the pressure-conveying line 42 supplied by the turbine 41 is present, on the same side as the rear face 2.2 of the valve shutter 2, so that the pressure prevailing therein can be exerted on the rear face 2.2 of the valve shutter 2, as already explained, here again making it possible to reduce the electrical power consumption of the coil 30.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A medical ventilator (100) comprising:
a motorized turbine (41) for supplying a gas,
computer (40) configured for operating the turbine (41),
a gas circuit (102) comprising an inspiratory branch (102.1) configured to carry the gas coming from the turbine (41) and an expiratory branch 102.2) in fluidic communication with the atmosphere, and
a PEP valve (1), arranged on the expiratory branch (102.2), comprising a valve shutter (2) comprising a front face (2.1) collaborating with a valve seat (3), and a rear face (2.2) on the opposite side to the front face (2.1),
characterized in that it further comprises:
an electromechanical-control device (20), operated by the computer (40), configured to act on the rear face (2.2) of the valve shutter (2) to push it towards the valve seat (3), and
a pressurizing chamber (22) in fluidic communication with a pressure-conveying line (42) supplied by the turbine (41), said pressurizing chamber (22) collaborating with the rear face (2.2) of the valve shutter (2).

2. The medical ventilator according to claim 1, characterized in that the electromechanical-control device (20) acts on the rear face (2.2) of the valve shutter (2) via an actuator (21).

3. The medical ventilator according to claim 1, characterized in that the pressurizing chamber (22) is arranged on the same side as the rear face (2.2) of the valve shutter (2) and is delimited by at least part of the surface of said rear face (2.2) of the valve shutter (2).

4. The medical ventilator according to claim 1, characterized in that the valve shutter (2) is made from a supple material.

5. The medical ventilator according to claim 1, characterized in that the valve shutter (2) comprises an internal chamber forming the pressurizing chamber (22).

6. The medical ventilator according to claim 1, characterized in that the electromechanical-control device (20) comprises a coil (30) and a magnet (31), these being mobile, one relative to the other.

7. The medical ventilator according to claim 6, characterized in that the coil (30) is controlled by the computer (40).

8. The medical ventilator according to claim 1, characterized in that the pressure-conveying line (42) comprises an atmospheric-venting duct (43) arranged between the turbine (41) and the PEP valve (1).

9. The medical ventilator according to claim 1, characterized in that the computer (40) comprises at least one processor.

10. The medical ventilator according to claim 1, characterized in that the medical ventilator comprises an electrical power-supply (106) supplying electrical power to the computer (40), to the motorized turbine (41), and/or to the electromechanical-control device (20).

11. The medical ventilator according to claim 1, characterized in that the electromechanical-control device (20) comprises an actuator (21) that acts against the rear face (2.2) of the valve shutter (2) to push it towards the valve seat (3).

12. The medical ventilator according to claim 11, characterized in that the actuator (21) is secured to said rear face (2.2) of the valve shutter (2).

13. The medical ventilator according to claim 2, characterized in that the actuator (21) is moved by a proportional electromagnet (VCA).

14. The medical ventilator according to one claim 1, characterized in that the electromechanical-control device (20) is at least partially arranged inside a casing (23) that can be secured to a PEP valve body (6).

15. The medical ventilator according to claim 14, characterized in that the actuator (21) is configured to move inside the casing (23).
